(19) United States
(12) Patent Application Publication
(10) Pub. No.: US 2023/0210793 A1
(43) Pub. Date: Jul. 6, 2023

(54) METHOD FOR RAPID INFUSION OF CARMUSTINE

(71) Applicant: Emcure Pharmaceuticals Limited, Pune (IN)

(72) Inventors: Deepak Pragjibhai GONDALIYA, Pune (IN); Hiren Pravinbhai PATEL, Pune (IN); Haresh Ishwarbhai PATEL, Pune (IN); Mukund Keshav GURJAR, Pune (IN)

(21) Appl. No.: 18/065,437

(22) Filed: Dec. 13, 2022

Related U.S. Application Data
(60) Provisional application No. 63/267,254, filed on Jan. 28, 2022.

(30) Foreign Application Priority Data
Dec. 31, 2021 (IN) 202123060432

(51) Int. Cl. A61K 31/17; A61K 9/00; A61K 45/06; A61K 9/08

(52) U.S. Cl. CPC A61K 31/17; A61K 9/0019; A61K 45/06; A61K 9/08

(57) ABSTRACT

The present invention relates to a method for rapid infusion of carmustine, for example, in 30 minutes or 1 hour.

This application claims the benefit of U.S. Provisional Application No. 63/267,254, filed Jan. 28, 2022, and Indian Patent Application No. 202123060432, filed Dec. 23, 2021, each of which is hereby incorporated by reference.

### FIELD OF THE INVENTION

The present invention relates to a method for rapid infusion of carmustine, for example, in 30 minutes or 1 hour.

### BACKGROUND OF THE INVENTION

Carmustine (bischloroethyl nitrosurea also known as BCNU) is a nitrosurea drug for the treatment of brain cancers owing to its ability to cross the blood-brain barrier and excellent activity against brain tumours.

Carmustine chemically known as 1,3-bis(2-chloroethyl)-1-nitrosourea (shown below) alkylates DNA, RNA and interferes with its synthesis and functions. It also binds and modifies (carbamoylates) glutathione reductase, which consequently leads to cell death.

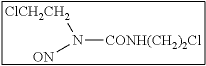

Carmustine is poorly soluble in water and is unstable in many formulations. For instance, carmustine gets readily hydrolyzed in water at pH >6. The solubility and stability issues of carmustine have been discussed previously. See, for example, Levin et al., Selective Cancer Therapeutics, 1989, 5(1), 33-35.

Carmustine is commercially available as a lyophilized 100 mg powder for injection under the trade name BiCNU® in single dose vials. See the March 2017 prescribing information for BiCNU®, which is hereby incorporated by reference. Ethanol (dehydrated alcohol) (3 mL) is co-packaged with the drug product as a sterile diluent for reconstitution. To prepare the drug for administration, three preparation steps need to be performed. First, the lyophilized carmustine is dissolved with the co-packed sterile dehydrated alcohol (3 mL) diluent. Second, the solution is further diluted with 27 mL of sterile water to form the reconstituted solution. Third, the reconstituted solution is further diluted with 5% Dextrose Injection, USP or Sodium Chloride Injection, USP (0.9% sodium chloride). This complicated preparation of carmustine solutions is time-consuming and can to lead to errors in preparation and dosing. The carmustine solution is administered only as a slow intravenous infusion over at least 2 hours. Administration of BiCNU® over a period of less than 2 hours can lead to pain and burning at the site of injection. According to the BiCNU® prescribing information, the rate of administration should not be more than 1.66 mg/m²/min.

### SUMMARY OF THE INVENTION

The present inventors surprisingly found that carmustine may be rapidly administered in a concentrated solution comprising propylene glycol and either an aqueous 0.9% sodium chloride solution or an aqueous 5% dextrose solution.

One embodiment is a method for rapidly administering carmustine to a patient in need thereof comprising administering by intravenous infusion an administrable solution of carmustine comprising (i) carmustine, (ii) propylene glycol, and (iii) an aqueous 0.9% sodium chloride solution or an aqueous 5% dextrose solution, wherein the concentration of carmustine in the administrable solution is up to about 3.1 mg/mL. The infusion is performed over less than 2 hours, for instance, about 30 minutes to about 1 hour (e.g., about 30 minutes or about 1 hour). In one embodiment, the concentration of carmustine in the administrable solution is no more than about 3.06 mg/mL. The administrable solution is preferably free or substantially free of ethanol. This method results in reduced time a patient needs to spend attached to an infusion and avoids the negative effects of being intoxicated due to ethanol present in BiCNU®.

Another embodiment is a method for rapidly administering carmustine to a patient in need thereof comprising administering by intravenous infusion over about 30 minutes to about 1 hour an administrable solution of carmustine consisting of (i) from about 2.8 to about 3.1 mg/mL (e.g., about 3.06 mg/mL) carmustine, (ii) propylene glycol, wherein the amount of propylene glycol is about 3 mL per 100 mg of carmustine, and (iii) an aqueous 0.9% sodium chloride solution or an aqueous 5% dextrose solution.

The administrable solution may be prepared by (a) dissolving lyophilized carmustine in sterile propylene glycol to form a reconstituted solution, wherein the amount of propylene glycol is 3 mL per 100 mg of carmustine; and (b) diluting the reconstituted solution with an aqueous 0.9% sodium chloride solution or an aqueous 5% dextrose solution to obtain the administrable solution having a carmustine concentration of from about 2.8 to about 3.1 mg/mL (e.g., about 3.06 mg/mL). In one embodiment, prior to dissolving the lyophilized carmustine in the propylene glycol, (i) the lyophilized carmustine and propylene glycol are stored in separate vials at 2-8° C. and (ii) the propylene glycol is allowed to attain room temperature just prior to dissolving the lyophilized carmustine in the propylene glycol. In another embodiment, prior to dissolving the lyophilized carmustine in the propylene glycol, (i) the lyophilized carmustine and propylene glycol are stored in separate vials at 2-8° C. and (ii) the vials are allowed to attain room temperature just prior to dissolving the lyophilized carmustine in the propylene glycol. The propylene glycol may be aseptically removed from its vial with a sterile syringe having a needle below 22 gauge and injected into the vial containing the lyophilized carmustine.

The reconstituted solution may contain at least 90% of the initial carmustine after storage at 2-8° C. for up to 480 hours. In one embodiment, the reconstituted solution is stored at 2-8° C. for up to 480 hours prior to step (b), and after storage at 2-8° C. and prior to performing step (b), the reconstituted solution is examined for crystal formation and if crystals are observed, they are re-dissolved by warming the reconstituted solution to room temperature with agitation. In a preferred embodiment, the reconstituted solution is protected from light during storage.

In another embodiment, (i) the reconstituted solution is stored at 2-8° C. for up to 24 hours or at room temperature for up to 8 hours and protected from light prior to step (b), (ii) optionally, after storage and prior to performing step (b), the reconstituted solution is examined for crystal formation and if crystals are observed, they are re-dissolved by warming the re-constituted solution to room temperature with agitation, and (iii) the administrable solution is stored under normal room fluorescent light at 2-8° C. for up to 24 hours and subsequently at room temperature for up to 6 hours prior to administration by intravenous infusion.

In one embodiment, step (b) is performed within 48 hours of the reconstituted solution being prepared.

The administrable solution may have a pH of about 4.2 to 4.8. The administrable solution may have an osmolarity of about 1900 to about 2000 mOsmol/L.

In one embodiment, the patient is administered about 300 mg/m² to about 800 mg/m² carmustine.

Another embodiment is a kit comprising a product vial containing lyophilized carmustine and a diluent vial containing a non-aqueous diluent (preferably propylene glycol). Preferably, the product vial contains only lyophilized carmustine. In one embodiment, the lyophilized carmustine does not contain a bulking agent, such as mannitol. Preferably, the diluent vial only contains the ethanol-free non-aqueous diluent (preferably propylene glycol). The product vial may contain 50-600 mg (e.g., 50-500 mg, 50-200 mg, 300-600 mg, 250-350 mg, or 450-550 mg) of lyophilized carmustine, and the diluent vial may contain 1-18 mL (e.g., 1-15 mL) of the ethanol-free non-aqueous diluent (e.g., propylene glycol). In a preferred embodiment, the product vial contains 33.33 mg of lyophilized carmustine for each mL of propylene glycol in the diluent vial. In one preferred embodiment, the product vial contains 100 mg of lyophilized carmustine and the diluent vial contains 3 mL of ethanol-free non-aqueous diluent, preferably propylene glycol (more preferably, sterile propylene glycol). In another preferred embodiment, the product vial contains 50 mg of lyophilized carmustine and the diluent vial contains 1.5 mL of ethanol-free non-aqueous diluent, preferably propylene glycol (more preferably, sterile propylene glycol). In yet another embodiment, the product vial contains 300 mg of lyophilized carmustine and the diluent vial contains 9 mL of ethanol-free non-aqueous diluent, preferably propylene glycol (more preferably, sterile propylene glycol). In yet another embodiment, the product vial contains 500 mg of lyophilized carmustine and the diluent vial contains 15 mL of ethanol-free non-aqueous diluent, preferably propylene glycol (more preferably, sterile propylene glycol). The use of vials containing greater amounts of carmustine and propylene glycol results in fewer opportunities for errors in preparing dilutions, including fewer needle pricks.

Another embodiment is a method of preparing an administrable solution of carmustine comprising (a) dissolving lyophilized carmustine in an ethanol-free, non-aqueous diluent (e.g., propylene glycol) to form a reconstituted solution, and (b) diluting the reconstituted solution with an aqueous 0.9% sodium chloride solution (preferably Sodium Chloride Injection, USP) or an aqueous 5% dextrose solution (preferably 5% Dextrose Injection, USP) to obtain the administrable solution. This method includes a single reconstitution step unlike the current procedure required for BiCNU® which includes two steps to reconstitute the carmustine (i.e., dissolution in 3 mL of ethanol followed by further dissolution in 27 mL of water). The reconstituted solution of the present invention has superior stability compared to reconstitution with 3 mL of ethanol.

In one embodiment, the administrable solution is prepared by (a) dissolving 50-600 mg (e.g., 50-500 mg, 300-500 mg, 300-600 mg, 50 mg, 100 mg, 300 mg, or 500 mg) of lyophilized carmustine in 1-18 mL (e.g., 1-15 mL) (e.g., 3 mL per 100 mg carmustine) of propylene glycol (e.g., sterile propylene glycol or propylene glycol USP) to form a reconstituted solution, and (b) diluting the reconstituted solution with an aqueous 0.9% sodium chloride solution or an aqueous 5% dextrose solution to obtain the administrable solution. In one embodiment, step (a) includes dissolving 300 mg of lyophilized carmustine in 9 mL of propylene glycol. In another embodiment, step (a) includes dissolving 500 mg of lyophilized carmustine in 15 mL of propylene glycol. In yet another embodiment, step (a) includes dissolving 100 mg of lyophilized carmustine in 3 mL of propylene glycol. In yet another embodiment, step (a) includes dissolving 50 mg of lyophilized carmustine in 1.5 mL of propylene glycol. In one embodiment, prior to administration, a total of 600-1200 mg of carmustine is prepared, for example, by separately dissolving at least one 300 or 500 mg of lyophilized carmustine in propylene glycol and 50, 100, 300, or 500 mg of lyophilized carmustine in propylene glycol. Prior to the present invention, when high amounts of carmustine were administered (e.g., at least 300 mg/m² or at least 600 mg), the ethanol diluent necessitated inpatient treatment or an increased post-infusion observation period. With the propylene glycol diluent, carmustine administration even at high doses does not require inpatient treatment or an increased post-infusion observation period (for example, treatment can be performed on an outpatient basis) thereby reducing the cost of treatment.

In a preferred embodiment, the reconstituted solution is stable (≥90% of carmustine remaining) after storage at 2-8° C. for up to 480 or 720 hours or at 25° C.±2° C. for 24 or 48 hours. Step (b), for example, may include diluting the reconstituted solution with an aqueous 0.9% sodium chloride solution (preferably Sodium Chloride Injection, USP) or an aqueous 5% dextrose solution (preferably 5% Dextrose Injection, USP) to a concentration of no more than about 3.1 mg/mL (e.g., no more than about 3.06 mg/mL or from about 0.5 to about 3.06 mg/mL). Step (b) is preferably performed within 480 hours (or 720 hours) of the reconstituted solution being prepared, where the reconstituted solution is stored at 2-8° C. After storage at 2-8° C. and prior to performing step (b), the reconstituted solution is preferably examined for crystal formation and if crystals are observed, they may be re-dissolved by warming the reconstituted solution to room temperature optionally with agitation. When the reconstituted solution is stored at room temperature, step (b) is preferably performed within 48 hours of the reconstituted solution being prepared. For instance, step (b) may be performed more than 24 hours but within 480 hours, or more than 24 hours but within 48 hours after the reconstituted solution is prepared. The lyophilized carmustine and the ethanol-free, non-aqueous diluent may be from a kit as described herein. In one preferred embodiment, the administrable solution has a pH in the range of about 4.2 to 4.8 (e.g., about 4.3 to about 4.6) and an osmolarity in the range of about 1800 to about 2100 mOsmol/L (e.g., about 1900 to about 2000 mOsmol/L).

Yet another embodiment is a method for administering carmustine to a patient in need thereof by administering by intravenous infusion over less than 2 hours (and preferably over about 30 minutes to about 1 hour) an administrable solution of carmustine having a carmustine concentration of no more than about 3.1 mg/mL, where the administrable solution is prepared from a kit comprising a product vial containing about 100, 200, 300, 400, 500, or 600 mg of lyophilized carmustine and a diluent vial containing about 3-18 mL of sterile propylene glycol (and preferably 3 mL of sterile propylene glycol per 100 mg lyophilized carmustine), and the kit is stored at 2-8° C. The administrable solution is prepared by:
(a) allowing the diluent vial to attain room temperature,
(b) aseptically removing the 3 mL of propylene glycol from the diluent vial (preferably with a below 22 gauge needle), injecting it into the product vial containing 100 mg of lyophilized carmustine, and shaking the product vial to dissolve the lyophilized carmustine to form a reconstituted solution,
(c) optionally, storing the reconstituted solution and prior to performing step
(d), the stored reconstituted solution is examined for crystal formation and if crystals are observed, they are re-dissolved by warming the reconstituted solution to room temperature with agitation, and
(d) diluting the reconstituted solution with an aqueous 0.9% sodium chloride solution or an aqueous 5% dextrose solution to obtain the administrable solution having a carmustine concentration of no more than about 3.1 mg/mL (e.g., no more than about 3.06 mg/mL or from about 0.5 to about 3.06 mg/mL).

Yet another embodiment is a method of administering carmustine comprising intravenously administering an administrable carmustine solution as described herein to a patient in need thereof over a period of less than 2 hours (e.g., about 30 minutes or about 1 hour). The administrable carmustine solution may be prepared as described herein.

In another embodiment, the rate of administration of the intravenous infusion is no more than 26.6 mg/m²/min. In yet another embodiment, the rate of administration of the intravenous infusion is no more than 26.6 mg/m²/min. In one embodiment, the rate of administration ranges from about 10 to about 26.6 mg/m²/min. For instance, the rate of administration of the intravenous infusion can be about 13.3 mg/m²/min or 26.6 mg/m²/min.

Yet another embodiment is a method of treating cancer in a patient in need thereof by intravenously administering over less than 2 hours (e.g., over about 30 minutes or about 1 hour) an administrable carmustine solution as described herein to the patient. The administrable carmustine solution may be prepared as described herein. The patient may be suffering from brain tumors glioblastoma, brainstem glioma, medulloblastoma, astrocytoma, ependymoma, metastatic brain tumors, multiple myeloma, relapsed or refractory Hodgkin's lymphoma, or relapsed or refractory Non-Hodgkin's lymphomas.

Yet another embodiment is a method for high-dose conditioning treatment of a patient with carmustine comprising administering to the patient an administrable carmustine solution as described herein to the patient with at least one other chemotherapeutic agent. The administrable carmustine solution may be prepared as described herein.

In one embodiment, the method comprises administering the carmustine as part of a BEAM regimen, that is, carmustine (referred to based on the trademark BiCNU® in the acronym BEAM) is administered with Etoposide, Ara-C (cytarabine) and Melphalan. For instance, one regimen can include administering 300 to 800 mg/m² carmustine on day −6 (six days prior to stem cell transplantation (SCT)), 100 or 150 mg/m² etoposide intravenously Q12H (every 12 hours) on day −5 to −2 (8 total doses), and 200 mg/m² cytarabine intravenously Q12H on days −5 to −2 (8 total doses) and 140 mg/m² melphalan on day −1. Stem cells are administered on day 0. In another embodiment, carmustine is administered at about 300 mg/m² in a BEAM regimen.

In another embodiment, the method comprises administering the carmustine as part of a CBV regimen, that is, Cyclophosphamide, carmustine (referred to based on the trademark BiCNU® in the acronym CBV) and etoposide (referred to based on its name VP-16). For instance, one regimen can include administering 450-800 mg/m² carmustine (or 450-600 mg/m² carmustine) on day −7, 900-1600 mg/m² etoposide on day −6 to −4 (Q12H) and 1.8 g/m² cyclophosphamide on day −3 to −2. Stem cells are administered on day 0. In another embodiment, carmustine is administered at about 600 mg/m² in a CBV regimen. In yet another embodiment, carmustine is administered at about 450 mg/m² in a CBV regimen.

In yet another embodiment, the method comprises administering the carmustine as part of a BEAC regimen, that is carmustine (referred to based on the trademark BiCNU® in the acronym BEAM) is administered with Etoposide, Ara-C (cytarabine) and Cyclophosphamide. For instance, one regimen can include administering 300 mg/m² carmustine on day −5, 800 mg/m² etoposide on day −4 to −2 (Q12H), 800 mg/m² cytarabine on day −4 to −2 (Q12H, 1600 mg/m²/day) and 140 mg/kg cyclophosphamide on day −6 and −5. Stem cells are administered on day 0.

In yet another embodiment, any of the methods described herein is performed prior to stem cell transplantation (SCT), for example, in a patient with relapsed and/or refractory lymphoma, such as relapsed/refractory (R/R) non-Hodgkin lymphoma (NHL).

Chemotherapeutic agents include, but are not limited to, alkylating agents (including, but not limited to, cyclophosphamide, ifosfamide, busulfan, chlorambucil, melphalan, temozolomide, cisplatin, carboplatin, and oxaliplatin), topoisomerase I inhibitors (such as irinotecan and topotecan), topoisomerase II inhibitors (such as etoposide, teniposide, doxorubicin, daunorubicin, and idarubicin), mitotic inhibitors (such as vincristine, vinblastine, and taxanes (e.g., docetaxel and paclitaxel)), antifolates (such as methotrexate and pemetrexed), pyrimidine antagonists (such as cytarabine, 5-fluorouruacil, gemcitabine, and capecitabine), purine analogs (such as 6-mercaptopurine (6-MP), azathioprine (prodrug for 6-MP), and cladribine), purine antagonists (such as fludarabine), ribonucleotide reductase inhibitors (such as hydroxyurea (hydroxycarbamide)), bleomycin, actinomycin D, mitomycin, L-asparaginase, proteasome inhibitors (such as bortezomib), and tyrosine kinase inhibitors (such as imatinib and erlotinib).

In one embodiment of any of the methods described herein, the patient receives carmustine in a regimen with etoposide, cytarabine, and melphalan. For instance, the patient may receive carmustine, etoposide, cytarabine, and melphalan as a conditioning regimen for autologous hematopoietic cell transplantation. In one embodiment, the patient suffers from relapsed or refractory non-Hodgkin lymphoma or Hodgkin lymphoma.

### DETAILED DESCRIPTION OF THE INVENTION

The terms “ethanol” and “dehydrated alcohol” are used synonymously throughout the specification.

The U.S. Pharmacopeia, USP 42-NF 37 (2019) is hereby incorporated by reference, including the entries for Sodium Chloride Injection, USP, 5% Dextrose Injection, USP.

Kit

One embodiment is a kit comprising a product vial containing lyophilized carmustine and a diluent vial containing ethanol-free non-aqueous diluent. Preferably, the product vial contains only lyophilized carmustine. In one embodiment, the lyophilized carmustine does not contain a bulking agent. The amount of lyophilized carmustine in product vial may vary from about 2 mg/vial to about 500 or 600 mg/vial, preferably 50 mg/vial, 100 mg/vial, 300 mg/vial, 500 mg/vial, and 600 mg/vial. The lyophilized carmustine, which typically is in the form of a powder, may be prepared by methods known in the art, such as those described in U.S. Patent Publication No. 2016/0136116, which is incorporated by reference.

Preferably, the diluent vial only contains the ethanol-free non-aqueous diluent (preferably propylene glycol). Suitable ethanol free non-aqueous diluents include, but are not limited to, aliphatic amides (such as N,N-dimethylacetamide and N-hydroxy-2-ethyl-lactamide), glycols and polyalcohols (such as propylene glycol and glycerine), esters of polyalcohols (such as diacetine (glyceryl diacetate), triacetine (glyceryl triacetate)), polyglycols and polyethers (such as propylene glycol methyl ethers), transcutol, dioxolanes (such as isopropylidene glycerine), N-methylpyrrolidone, or any combination of any of the foregoing. According to one preferred embodiment, the ethanol-free non-aqueous diluent is propylene glycol, N,N-dimethylacetamide, transcutol, or methylpyrrolidone. The ethanol-free non-aqueous diluent is preferably sterile. A more preferred ethanol-free non-aqueous diluent is propylene glycol, such as sterile propylene glycol or propylene glycol USP.

The amount of ethanol-free non-aqueous diluent in the diluent vial may vary from between 1 ml and 20 ml. Preferably the amount of non-aqueous diluent is 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, or 18 ml. Preferably, the amount of the non-aqueous diluent propylene glycol is 3 mL per 100 mg of lyophilized carmustine in the product vial.

In one embodiment, the product vial contains 50-600 mg (e.g., 50-500 mg) of lyophilized carmustine, and the diluent vial contains 1-18 mL (e.g., 1-15 mL) (e.g., 3 mL per 100 mg of carmustine) of the ethanol-free non-aqueous diluent (e.g., propylene glycol). In a preferred embodiment, the product vial contains 100, 200, 250, 300, 400, 500, or 600 mg of lyophilized carmustine and the diluent vial contains 3 mL of ethanol-free non-aqueous diluent, preferably propylene glycol (more preferably, sterile propylene glycol or propylene glycol USP) per 100 mg of lyophilized carmustine in the product vial.

The vials are preferably made of glass or polypropylene (such as polypropylene which is polyvinyl chloride (PVC) free and di-2-ethylhexyl phthalate (DEHP) free). The vials are preferably not made of (and do not contain) polyvinyl chloride or DEHP.

In a preferred embodiment, the product vial is stored at 2-8° C. In another preferred embodiment, the product vial and diluent vial are stored at 2-8° C.

Preparation

In another embodiment, the present invention provides a single-step reconstitution procedure for carmustine injection wherein the lyophilized carmustine, such as from the product vial, is reconstituted with a specified amount of the ethanol-free non-aqueous diluent (preferably propylene glycol), such as from the diluent vial.

This reconstitution procedure of the present invention is advantageous over the current procedure used for BiCNU® as it requires a single-step dilution with an ethanol-free non-aqueous diluent. In other words, the additional step of dilution with 27 mL of water for injection as described in the current package insert for BiCNU® is eliminated.

Prior to reconstitution, the diluent vial may be allowed to attain room temperature, for example, by removal from a refrigerator (where it is stored at 2-8° C.). In one embodiment, both the product vial and diluent vial are removed from a refrigerator (where they are stored at 2-8° C.) and allowed to attain room temperature. In one embodiment, the propylene glycol is removed from the diluent vial using an appropriate needle (e.g., a 22 gauge needle or a needle below 22 gauge). In one preferred embodiment, the needle is below 22 gauge. In one embodiment, the propylene glycol is aseptically removed from the diluent vial with a sterile syringe and injected into the product vial containing carmustine. The product vial may be gently shaken to dissolve the carmustine.

The typical two-step reconstitution procedure for the current BiCNU® product (as per its package insert) is as described below:
1) Aseptically removing 3 mL of ethanol diluent from the diluent vial using a sterile syringe and injecting it into the product vial containing the lyophilized carmustine, followed by gentle shaking to obtain a clear solution, and
2) Aseptically adding 27 mL of sterile water for injection into the solution of step (1), followed by gentle shaking to obtain a clear solution.

The single-step reconstitution procedure of the present invention, in contrast, can be as described below:
1) Aseptically removing an appropriate quantity (e.g. 3 mL) of ethanol-free non-aqueous diluent (e.g., propylene glycol) from the diluent vial using a sterile syringe and injecting it into the product vial containing the lyophilized carmustine, followed by gentle shaking to obtain a clear solution.

Preferably, the diluent (preferably propylene glycol) is allowed to attain room temperature before it is aseptically removed from its vial and injected into the product vial. Preferably, the lyophilized carmustine dissolves in the propylene glycol within 3 minutes and more preferably within 2 minutes.

In one embodiment, the reconstituted carmustine solution has a concentration of about 33.33 mg/mL of carmustine.

Prior to administration, the reconstituted carmustine solution may be further admixed with 0.9% sodium chloride injection or 5% dextrose injection to form an administrable solution. The reconstituted carmustine solution is further diluted with 0.9% sodium chloride injection or 5% dextrose injection to a carmustine concentration of no more than about 3.1 mg/mL. In one embodiment, the reconstituted carmustine solution is further diluted with 250 mL of 0.9% sodium chloride injection. In another embodiment, the reconstituted carmustine solution is further diluted with 250 mL of 5% dextrose injection. In yet another embodiment, the reconstituted carmustine solution is further diluted with 500 mL of 0.9% sodium chloride injection. In yet another embodiment, the reconstituted carmustine solution is further diluted with 500 mL of 5% dextrose injection.

The reconstituted carmustine solution may be stored at room temperature or at 2-8° C. prior to being admixed with the 0.9% sodium chloride injection or 5% dextrose injection. The admixing step is preferably performed within 480 hours of the reconstituted solution being prepared, where the reconstituted solution is stored at 2-8° C. After storage at 2-8° C. and prior to being admixed, the reconstituted solution is preferably examined for crystal formation and if crystals are observed, they may be re-dissolved by warming the re-constituted solution to room temperature optionally with agitation. When the reconstituted solution is stored at room temperature, the admixing step is preferably performed within 48 hours of the reconstituted solution being prepared. For instance, the admixing step may be performed more than 24 hours but within 480 hours, or more than 24 hours but within 48 hours after the reconstituted solution is prepared.

The reconstituted solution and/or administrable solution may be stored in a glass or polypropylene container (such as a polypropylene container which is polyvinyl chloride (PVC) free and di-2-ethylhexyl phthalate (DEHP) free). These solutions are preferably not stored in a polyvinyl chloride or DEHP container.

The administrable solution may be a faint yellow colour with a pH in the range of about 4 to about 5 and osmolarity in the range of about 1800 to about 2100 mOsmol/L. In a preferred embodiment, the administrable solution has a pH of about 4.2 to about 4.8 and osmolarity of about 1900 to about 2000 mOsmol/L mOsmol/L.

The administrable solution can have a concentration of about 2.8 to about 3.1 mg/mL carmustine. In a preferred embodiment, the administrable solution has a concentration of no more than about 3.06 mg/mL. In one embodiment, the administrable solution has a concentration of no more than about 3.1 mg/mL. The administrable solution may have a concentration of at least about 0.5 mg/mL, 0.6 mg/mL, 0.7 mg/mL, 0.8 mg/mL, 0.9 mg/mL, or 1.0 mg/mL and a concentration of no more than about 3.1 mg/mL or 3.06 mg/mL. In another embodiment, the administrable solution has a concentration of about 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.1 mg/mL, 2.2 mg/mL, 2.3 mg/mL, 2.4 mg/mL, 2.5 mg/mL, 2.6 mg/mL, 2.7 mg/mL, 2.8 mg/mL, 2.9 mg/mL, 3.0 mg/mL, or 3.1 mg/mL. The concentration of the administrable solution can be determined based on the desired dose. The desired dose can, for example, be based on the body surface area of the patient (e.g., mg/m²). The appropriate amount of reconstituted carmustine solution can be added to a 250 or 500 mL intravenous solution (such as an aqueous 0.9% sodium chloride solution or an aqueous 5% dextrose solution). The preparation of the reconstituted carmustine solution and administrable solution can be prepared by one skilled in the art, such as a pharmacist.

In one embodiment, a sufficient amount of the administrable solution is administered over less than 2 hours (e.g., from about 30 minutes to about 1 hour, or about 30 minutes or about 1 hour) to provide the patient with from about 200 to about 1,700 mg of carmustine, such as from about 300 to about 1,700 mg of carmustine, from about 300 to about 750 mg of carmustine, or from about 600 to about 1,700 mg of carmustine.

In one embodiment, the patient is administered a dose of carmustine of about 300 to about 800 mg/m² carmustine. In another embodiment, the patient is administered a dose of carmustine of about 300 mg/m² carmustine. In yet another embodiment, the patient is administered a dose of carmustine of about 450 to about 800 mg/m² carmustine. In yet another embodiment, the patient is administered a dose of carmustine of about 450 to about 600 mg/m² carmustine. In yet another embodiment, the patient is administered a dose of carmustine of about 75 to about 100 mg/m² carmustine. In yet another embodiment, the patient is administered a dose of carmustine of about 150 to about 200 mg/m² carmustine.

The administrable solution may contain impurities associated with (i) carmustine, (ii) propylene glycol (e.g., Propylene Glycol, USP), and (iii) aqueous 0.9% sodium chloride solution (e.g., Sodium Chloride Injection, USP) or aqueous 5% dextrose solution (e.g., 5% Dextrose Injection, USP).

In another embodiment, the reconstituted carmustine solution is stable.

As used herein, a “stable” reconstituted carmustine solution means no aggregation was observed when stored at 2 to 8° C. (long-term storage condition) and 25° C.±2° C. (accelerated storage condition) for an appropriate time and where the assay of carmustine is ≥90%.

The carmustine content after storage is determined by high performance liquid chromatography (HPLC method). HPLC was used for performing the assay studies described in the examples below.

The reconstituted carmustine solution is stable for up to 720 hours (e.g., for up to 480 hours) when stored at 2° C.-8° C. and for up to 48 hours when stored at 25° C.±2° C. In contrast, the reconstituted carmustine solution of the reference product is stable only under refrigerated conditions (2° C.-8° C.) for up to 96 hours.

The stability of the admixed carmustine solution was also performed separately at 2° C. to 8° C. (long-term storage condition) for an appropriate time, 25° C.±2° C. (accelerated storage condition) for appropriate time and 2° C. to 8° C. for appropriate time followed by 25° C.±2° C. for appropriate time.

Based on the results shown in Table 1, it was concluded that the administrable solution prepared with 0.9% sodium chloride solution in a glass or polypropylene container is stable (≥90% carmustine remaining) for 24 hours at 2-8° C. followed by up to 6 hours (25° C.±2° C.). This administrable solution in a glass or polypropylene container is also stable for up to 48 hours at 2 to 8° C. This administrable solution in a glass or polypropylene container is stable for up to 8 hours at 25° C.±2° C.

The administrable solution prepared with 5% dextrose injection in a glass or polypropylene container is stable for up to 24 hours at 2 to 8° C. followed by up to 12 hours at 25° C.±2° C. This administration solution in a glass or polypropylene container is also stable for up to 48 hours at 2 to 8° C. followed by up to 6 hours at 25° C.±2° C. This administration solution in a glass or polypropylene container is stable for up to 8 hours at 25° C.±2° C.

Administration

The carmustine administrable solution may be rapidly administered to a patient (e.g., a human patient) by intravenous infusion over less than two hours. Preferably, the infusion is performed over less than 2 hours, for instance, about 30 minutes to about 1 hour. In one embodiment, the infusion is performed over about 30 minutes. In another embodiment, the infusion is performed over about 1 hour. In one embodiment, no more than 550.5 mL of the administrable solution is administered per 30 minute interval. In another embodiment, from about 500 to about 550.5 mL of the administrable solution is administered per 30 minute interval. In yet another embodiment, from about 500 to about 550.5 mL of the administrable solution is administered over from about 30 minutes to about 1 hour. In yet another embodiment, from about 500 to about 550.5 mL of the administrable solution is administered over about 30 minutes. In yet another embodiment, from about 500 to about 550.5 mL of the administrable solution is administered over about 1 hour. In yet another embodiment, from about 250 to about 275.3 mL of the administrable solution is administered per 30 minute interval. In yet another embodiment, from about 250 to about 275.3 mL of the administrable solution is administered over from about 30 minutes to about 1 hour. In yet another embodiment, from about 250 to about 275.3 mL of the administrable solution is administered over about 30 minutes. In yet another embodiment, from about 250 to about 275.3 mL of the administrable solution is administered over about 1 hour.

In one embodiment, the injected area is monitored during the administration.

In another embodiment, the rate of administration of the intravenous infusion is no more than 26.6 mg/m²/min. In yet another embodiment, the rate of administration of the intravenous infusion is no more than 26.6 mg/m²/min. In one embodiment, the rate of administration ranges from about 10 to about 26.6 mg/m²/min. For instance, the rate of administration of the intravenous infusion can be about 13.3 mg/m²/min or 26.6 mg/m²/min.

The patient may suffer from cancer.

In one embodiment, the carmustine administrable solution may be administered to a patient to treat brain tumors glioblastoma, brainstem glioma, medulloblastoma, astrocytoma, ependymoma, metastatic brain tumors, multiple myeloma, relapsed or refractory Hodgkin's lymphoma, or relapsed or refractory Non-Hodgkin's lymphomas.

In one embodiment, the carmustine administrable solution is administered to a patient as a single agent or in a combination therapy (such as with other chemotherapeutic agents) to treat (i) brain tumors glioblastoma, brainstem glioma, medulloblastoma, astrocytoma, ependymoma, or metastatic brain tumors, (ii) multiple myeloma in combination with prednisone, (iii) relapsed or refractory Hodgkin's lymphoma in combination with other approved drugs (such as chemotherapeutic agents), or (iv) relapsed or refractory Non-Hodgkin's lymphomas in combination with other approved drugs (such as chemotherapeutic agents).

The carmustine administrable solution may be administered as a single agent in previously untreated patients at a dose of 150 to 200 mg/m² carmustine intravenously every 6 weeks. The carmustine administrable solution may be administered as a single dose or divided into daily injections such as 75 to 100 mg/m² on two successive days. The dose may be lowered when the carmustine administrable solution is used with other myelosuppressive drugs or in patients in whom bone marrow reserve is depleted. The carmustine administrable solution may be administered for the duration according to the established regimen. In one embodiment, the patient is premedicated before each dose with antiemetics.

In another embodiment, the administrable solution is administered (e.g., as a single agent) in a patient (such as a previously untreated patient) at a dose of 300 to 800 mg/m² (e.g., 450 to 800 mg/m² or 450 to 600 mg/m²) carmustine intravenously. In yet another embodiment, the administrable solution is administered as part of a BEAM treatment in a patient (such as a previously untreated patient) at a dose of 300 to 800 mg/m² (e.g., 450 to 800 mg/m² or 450 to 600 mg/m²) carmustine intravenously.

The dosing (after the initial dose) may be adjusted according to the hematologic response of the patient to the preceding dose. In one embodiment, the patient is dosed as follows:

| | | Percentage of |
|---|---|---|
| Nadir After Prior Dose | | Prior Dose to |
| Leukocytes/mm³ | Platelets/mm³ | be Given |
| >4000 | >100,000 | 100% |
| 3000-3999 | 75,000-99,999 | 100% |
| 2000-2999 | 25,000-74,999 | 70% |
| <2000 | <25,000 | 50% |

The hematologic toxicity can be delayed and cumulative. In one embodiment, the patient's blood counts are monitored weekly. In another embodiment, a repeat course of the carmustine administrable solution is not administered until circulating blood elements have returned to acceptable levels (platelets above 100 Gi/L, leukocytes above 4 Gi/L and absolute neutrophil count above 1 Gi/L). In yet another embodiment, the interval between courses is 6 weeks.

In one embodiment, the carmustine administrable solution is administered in a high-dose conditioning treatment. One embodiment is a method for high-dose conditioning treatment of a patient with carmustine comprising administering to the patient an administrable carmustine solution as described herein to the patient with at least one other chemotherapeutic agent. The administrable carmustine solution may be prepared as described herein. In one embodiment, the method comprises administering the carmustine as part of a BEAM regimen, that is, carmustine (referred to based on the trademark BiCNU® in the acronym BEAM) is administered with Etoposide, Ara-C (cytarabine) and Melphalan. In another embodiment, the method comprises administering the carmustine as part of a CBV regimen, that is, Cyclophosphamide, carmustine (referred to based on the trademark BiCNU® in the acronym CBV) and etoposide (referred to based on its name VP-16). In yet another embodiment, the method comprises administering the carmustine as part of a BEAC regimen, that is carmustine (referred to based on the trademark BiCNU® in the acronym BEAM) is administered with Etoposide, Ara-C (cytarabine) and Cyclophosphamide. In yet another embodiment, any of the methods described herein is performed prior to stem cell transplantation (SCT) (e.g., hematopoietic stem cell transplantation or autologous stem cell transplantation), for example, in a patient with relapsed and/or refractory lymphoma, such as relapsed/refractory (R/R) non-Hodgkin lymphoma (NHL).

In yet another embodiment, renal function is evaluated prior to administration and/or periodically during treatment. In one embodiment, carmustine treatment is discontinued if the creatinine clearance is less than 10 mL/min. In another embodiment, carmustine is not administered to patients with compromised renal function. In yet another embodiment, transaminases and bilirubin are monitored periodically during treatment.

The following examples further illustrate the invention but should not be construed as in any way limiting its scope. In particular, the processing conditions are merely exemplary and can be varied by one of ordinary skill in the art.

### EXAMPLES

In the examples and tables below, the following terms and abbreviations have the specified definitions.

“IA” refers to Impurity A.

“Impurity A” refers to 1,3-bis(2-chloroethyl)urea.

“IUUI” refers to an individual unspecified unidentified impurity.

“TI” refers to total impurities.

The content of carmustine and impurities was determined by high performance liquid chromatography (HPLC).

### Example 1

A product vial containing 100 mg lyophilized carmustine and a diluent vial containing 3.0 mL of sterile propylene glycol were removed from a refrigerator and allowed to attain room temperature. The 3 mL of propylene glycol was aseptically removed from the diluent vial using a sterile syringe and injected into the product vial containing the lyophilized carmustine. The product vial was gently shaken to form a clear solution. The reconstituted carmustine solution is stable (≥90% carmustine remaining) at 2-8° C. for 720 hours and at 25±2° C. for up to 48 hours.

The reconstituted carmustine solution was further admixed to bring its total volume to 500 mL with 0.9% sodium chloride injection or 5% dextrose injection to form a carmustine administrable solution for clinical use. The concentration of carmustine in the administrable solution was 3.06 mg/mL. The stability of the admixed carmustine administrable solution at (i) 2-8° C. for 24 hours followed by 25±2° C. for 12 hours, (ii) 2-8° C. for 48 hours followed by 25±2° C. for 12 hours, and (iii) 25±2° C. for 8 hours was evaluated. The results are provided in Tables 1 and 2 below.

TABLE 1
Assay of 3.06 mg/mL carmustine administrable solution with 0.9%
sodium chloride injection (NaCl) and 5% dextrose injection

| | Assay (%) | | | |
|---|---|---|---|---|
| | Glass | | Polypropylene | |
| Sampling Intervals and Storage | Container | | Container | |
| Condition | NaCl | Dextrose | NaCl | Dextrose |

Stored up to 24 hours at 2° C.-8° C. and further up to 12 hours at
25° C. ± 2° C.

| | | | | |
|---|---|---|---|---|
| Initial (0 hour) | 102.3 | 103.3 | 102.3 | 103.3 |
| 12 hrs. (2-8° C.) | 98.7 | 99.4 | 98.6 | 100.7 |
| 24 hrs. (2-8° C.) | 96.8 | 99.7 | 96.8 | 99.7 |
| 24 hrs. (2-8° C.) + 6 hrs. | 92.8 | 99.3 | 93.1 | 100.4 |
| (25° C. ± 2° C.) | | | | |
| 24 hrs. (2-8° C.) + 12 hrs. | 86.5 | 91.8 | 87.5 | 93.4 |
| (25°C ± 2° C.) | | | | |

Stored up to 48 hours at 2° C.-8° C. and further up to 12 hours at
25° C. ± 2° C.

| | | | | |
|---|---|---|---|---|
| Initial (0 hour) | 100.4 | 104.0 | 100.4 | 104.0 |
| 48 hrs. (2-8° C.) | 93.3 | 95.5 | 95.1 | 95.5 |
| 48 hrs. (2-8° C.) + 6 hrs. | 89.5 | 93.6 | 90.2 | 94.1 |
| (25° C. ± 2° C.) | | | | |
| 48 hrs. (2-8° C.) + 12 hrs. | 84.2 | 89.1 | 85.5 | 89.5 |
| (25° C. ± 2° C.) | | | | |

Stored up to 8 hours at 25° C. ± 2° C.

| | | | | |
|---|---|---|---|---|
| Initial (0 hour) | 101.6 | 101.4 | 101.6 | 101.4 |
| 4 hrs. (25° C. ± 2° C.) | 97.0 | 98.1 | 96.7 | 98.1 |
| 8 hrs. (25 ± 2° C.) | 92.5 | 94.0 | 92.4 | 93.6 |

TABLE 2
Impurity data of 3.06 mg/mL carmustine administrable solution with 0.9%
sodium chloride injection (NaCl) and 5% dextrose injection

| Sampling | Related Substance (%) | | | | | | | | | | | |
|---|---|---|---|---|---|---|---|---|---|---|---|---|
| intervals and | Glass Container | | | | | | Polypropylene Container | | | | | |
| storage | NaCl | | | Dextrose | | | NaCl | | | Dextrose | | |
| condition | IA | IUUI | TI | IA | IUUI | TI | IA | IUUI | TI | IA | IUUI | TI |

Stored at 24 Hrs. at 2° C.-8° C. and furt ler for 12 hrs. at 25° C. ± 2° C.

| | | | | | | | | | | | | |
|---|---|---|---|---|---|---|---|---|---|---|---|---|
| Initial (0) | 0.20 | 0.004 | 0.20 | 0.21 | BLQ | 0.21 | 0.20 | 0.004 | 0.20 | 0.21 | BLQ | 0.21 |
| 12 hrs (2-8° C.) | 0.19 | 0.009 | 0.20 | 0.10 | 0.029 | 0.14 | 0.20 | 0.008 | 0.20 | 0.18 | 0.034 | 0.23 |
| 24 hrs (2-8° C.) | 0.22 | 0.014 | 0.23 | 0.19 | 0.046 | 0.25 | 0.19 | 0.013 | 0.20 | 0.18 | 0.047 | 0.24 |
| 24 hrs (2-8° C.) + | 0.20 | 0.026 | 0.23 | 0.26 | 0.082 | 0.36 | 0.19 | 0.023 | 0.21 | 0.20 | 0.075 | 0.29 |
| 6 hrs (25° C. ± 2° C.) | | | | | | | | | | | | |
| 24 hrs (2-8° C.) + | 0.21 | 0.040 | 0.26 | 0.21 | 0.150 | 0.38 | 0.20 | 0.037 | 0.25 | 0.19 | 0.142 | 0.35 |
| 12 hrs (25° C. ± 2° C. | | | | | | | | | | | | |

Stored at 48 Hrs. at 2° C.-8° C. and further for 12 hrs. at 25° C. ± 2° C.

| | | | | | | | | | | | | |
|---|---|---|---|---|---|---|---|---|---|---|---|---|
| Initial (0 hour) | 0.19 | 0.005 | 0.20 | 0.23 | 0.010 | 0.24 | 0.19 | 0.005 | 0.20 | 0.23 | 0.010 | 0.24 |
| 48 hrs (2-8° C.) | 0.19 | 0.024 | 0.22 | 0.23 | 0.092 | 0.34 | 0.19 | 0.024 | 0.22 | 0.19 | 0.095 | 0.30 |
| 48 hrs (2-8° C.) + | 0.20 | 0.035 | 0.24 | 0.22 | 0.136 | 0.38 | 0.20 | 0.033 | 0.24 | 0.21 | 0.137 | 0.37 |
| 6 hrs (25° C. ± 2° C.) | | | | | | | | | | | | |
| 48 hrs (2-8° C.) + | 0.21 | 0.047 | 0.27 | 0.21 | 0.178 | 0.41 | 0.21 | 0.044 | 0.26 | 0.21 | 0.173 | 0.40 |
| 12 hrs (25° C. ± 2° C.) | | | | | | | | | | | | |

Stored at 8 hrs. at 25° C. ± 2° C.

| | | | | | | |
|---|---|---|---|---|---|---|
| Initial (0 hour) | 0.20 | BLQ | 0.20 | 0.20 | BLQ | 0.20 |
| 4 hrs. (25° C. ± 2° C.) | 0.17 | 0.036 | 0.23 | 0.19 | 0.037 | 0.25 |
| 8 hrs. (25° C. ± 2° C.) | 0.16 | 0.077 | 0.25 | 0.18 | 0.078 | 0.27 |

As shown by Table 1, the 3.06 mg/mL carmustine administrable solution prepared with 0.9% sodium chloride injection in a glass or polypropylene container is stable (≥90% carmustine remaining) for up to 24 hours at 2 to 8° C. followed by up to 6 hours at 25° C.±2° C. This administrable solution in a glass or polypropylene container is also stable for up to 48 hours at 2 to 8° C. This administrable solution in a glass or polypropylene container is stable for up to 8 hours at 25° C.±2° C.

The 3.06 mg/mL carmustine administrable solution prepared with 5% dextrose injection in a glass or polypropylene container is stable (≥90% carmustine remaining) for up to 24 hours at 2 to 8° C. followed by up to 12 hours at 25° C.±2° C. This administrable solution in a glass or polypropylene container is also stable for up to 48 hours at 2 to 8° C. followed by up to 6 hours at 25° C.±2° C. This administrable solution in a glass or polypropylene container is stable for up to 8 hours at 25° C.±2° C.

### Prophetic Example 2

This is a single center phase 2 study of the carmustine administration solution of Example 1 versus BiCNU® in the BEAM (carmustine, etoposide, Ara-C, and melphalan) high intensity conditioning regimen for autologous hematopoietic cell transplantation (autoHCT) in patients with relapsed or refractory non-Hodgkin lymphoma (NHL) and Hodgkin lymphoma (HL). This study is intended to include 46 to 49 participants. There will be an initial lead-in phase with up to 9 participants with Example 1, followed by a randomized portion to Arm A (n=25) with Example 1 or Arm B (n=15) with BiCNU® (2 hour infusion).

During the lead-in portion, the first cohort of 3 participants will receive Example 1 with a 2 hour infusion. If none of the first cohort have infusion related toxicities, the next cohort of 3 will receive Example 1 with a 1 hour infusion. If none of the cohort receiving the 1 hour infusion have infusion related toxicities, the randomization will open, and Arm A will have Example 1 with a 30 minute infusion. Infusion related toxicities will be monitored for the first 6 participants on Arm A with a 30 minute infusion. If two or more participants (out of 6 participants) receiving the 30 minute infusion have infusion-related toxicities, later Arm A participants will receive Example 1 with a 1 hour infusion.

If none of the 3 participants receiving Example 1 over a 2 hour infusion have infusion related toxicities, but subsequently one of three receiving Example 1 over a 1 hour infusion have infusion related toxicities, the 1 hour cohort will be expanded to 6. If 1 of the 6 participants receiving Example 1 over a 1 hour infusion have infusion related toxicities, the randomization will open, and Arm A will have Example 1 over a 1 hour infusion. If instead two or more out of 3 participants or two or more out of 6 participants receiving Example 1 over a 1 hour infusion have infusion related toxicities, the randomization will open, and Arm A will have Example 1 with a 2 hour infusion.

If one of 3 participants receiving Example 1 over a 2 hour infusion have infusion related toxicities, the 2 hour cohort will be expanded to 6. If one of the 6 participants receiving the 2 hour infusion has infusion related toxicities, the randomization will open, and Arm A will have Example 1 over a 2 hour infusion. If two or more out of 3 participants or two or more out of 6 participants receiving Example 1 over a 2 hour infusion have infusion related toxicities, the accrual will be stopped.

In addition, accrual will be held for review with potential study amendment if:
more than 1 participant with unacceptable toxicities within 30 days post autoHCT in the first 6 participants receiving Example 1 over a 2 hour or 1 hour infusion, or if the unacceptable toxicity rate is 20% or more with Example 1 thereafter (with 2 hour, 1 hour or 30 minute infusion combined).
infusion related toxicity rate is 15% or more on Arm A when there are more than 6 participants (excluding the first few participants on Arm A at 30 minute infusion if subsequently infusion was changed to 1 hour as discussed above).

### Objectives

### Primary Objectives

Evaluate the infusion related toxicities for Example 1 within 24 hours post infusion
Evaluate the unacceptable toxicities for Example 1 and BiCNU® from start of BEAM through Day 30 post-HCT

### Secondary Objectives

Evaluate the following toxicities potentially associated with the BiCNU® diluents for Example 1 and BiCNU®: flushing, hypotension, nausea, anxiety, confusion, depression, agitation, delirium, hemolysis, supraventricular tachycardia, and acidosis
Evaluate transplant related outcomes for Example 1 and BiCNU®: incidence of relapse/progression and non-relapse mortality (NRM) and time to neutrophil/platelet count recovery

### Endpoints

### Primary Endpoint(s)

Infusion related toxicity, which is defined as any of the following within 24 hours of Example 1 and BiCNU® infusion, with the exception of events that are deemed “unrelated” to protocol treatment:
any grade ≥3 Bearman toxicities (Bearman, S. I., et al., Regimen-related toxicity in patients undergoing bone marrow transplantation. J Clin Oncol, 1988. 6(10):1562-8)
grade ≥3 infusion related reactions according to CTCAE 5.0
any infusion interruption or a need to reduce infusion rate due to patient safety concerns

Unacceptable toxicity, which is defined as any of the following below plus any death, from start of BEAM through day 30 post autoHCT, with the exception of events that are deemed “unrelated” to protocol treatment.

| Scale | Organ (Toxicity) | Grade | Duration |
|---|---|---|---|
| Bearman | Cardiac | 3 or 4 | Any |
| | Bladder | 3 or 4 | Any |
| | Renal | 3 or 4 | Any |
| | Pulmonary | 3 or 4 | Any |
| | Hepatic | 3 or 4 | Any |
| | Central Nervous System | 3 or 4 | Any |
| | Stomatitis | 3 or 4 | Any |
| | Gastrointestinal | 3 or 4 | Any |
| NCI | Neutropenia, absolute neutrophil | 4 | >42 days |
| CTCAE 5.0 | count <500/mm³ | | |
| | Infusion related reaction | 3, 4 or 5 | Any |
| | Platelet count <25,000/mm³ | 4 | >42 days |
| | Nervous system disorders | 3, 4 or 5 | Any |

### Secondary Endpoints

Incidence of the following toxicities within 24 hours of BiCNU® and Example 1 infusion: flushing, hypotension, nausea, anxiety, confusion, depression, agitation, delirium, hemolysis, supraventricular tachycardia, and acidosis.

Incidence of non-relapse mortality and that of relapse/progression from start of BEAM through day 100 post autoHCT, time to neutrophil recovery and time to platelet recovery.

### Intervention Description

Patients who have completed at least 2 cycles of standard cytoreductive salvage chemotherapy followed by peripheral hematopoietic progenitor cell (HPC-A) collection of at least 2.0×10⁶ CD34 cells/kg will undergo treatment as follows:

| | |
|---|---|
| Day −6 | Example 1 300 mg/m² IV (Arm A or Lead-in) OR |
| | BiCNU ® 300 mg/m² IV (Arm B) |
| Day −5 to | Etoposide 200 mg/m² IV QD & Ara-C 200 mg/m² |
| −2 | IV BID for 4 days |
| Day −1 | Melphalan 140 mg/m² IV |
| Day 0 | Infusion of cryopreserved autologous HPC-A product |
| Day 5 | Start G-CSF (or biosimilar) 5 μg/kg/day (IV is the |
| | preferred route of administration) |

Patients will be followed for 24 hours post BiCNU® and Example 1 for infusion-related toxicities, 30 days post autoHCT for treatment-related toxicities, and 100 days post autoHCT for disease status, engraftment and non-relapse mortality.

### Main Inclusion Criteria

Adult (ages ≥18 years) patients
Histologically confirmed NHL or HL that is:
primary induction failure
early first relapse, or
second or subsequent relapse
Karnofsky performance status (PS)≥70%
Life expectancy ≥6 months
Adequate organ function
Patients will be enrolled after collection of at least 2.0×10⁶ CD34 cells/kg of autologous HPC-A by apheresis

### Main Exclusion Criteria

Prior autologous or allogeneic hematopoietic stem cell transplantation
Active Hepatitis B or C viral infection or Hepatitis B surface antigen positive
Positive Human Immunodeficiency Virus (HIV) antibody, patients with undetectable HIV viral load with CD4 ≥300 cells/μL are allowed
Pregnancy
Significant prior external beam dose-limiting radiation to a critical organ.

Persistent marrow involvement (>10%) with NHL or HL after salvage cytoreductive therapy and before stem cell mobilization

### Abbreviations

| Term | Meaning |
|---|---|
| BID | Twice a day |
| CTCAE | Common Terminology Criteria for Adverse Events |
| G-CSF | Granulocyte colony stimulating factor |
| HIV | Human Immunodeficiency Virus |
| HPC-A | Autologous hematopoietic progenitor cells |
| NCI | National Cancer Institute |
| QD | Daily |

All patents and other references cited herein are hereby incorporated by reference in their entireties.

1. A method for rapidly administering carmustine to a patient in need thereof comprising administering by intravenous infusion over about 30 minutes to about 1 hour an administrable solution of carmustine consisting of (i) up to about 3.1 mg/mL carmustine, (ii) propylene glycol, wherein the amount of propylene glycol is about 3 mL per 100 mg of carmustine, and (iii) an aqueous 0.9% sodium chloride solution or an aqueous 5% dextrose solution.

2. A method for rapidly administering carmustine to a patient in need thereof comprising administering by intravenous infusion an administrable solution of carmustine comprising (i) carmustine, (ii) propylene glycol, and (iii) an aqueous 0.9% sodium chloride solution or an aqueous 5% dextrose solution, wherein the concentration of carmustine in the administrable solution is from about 0.5 to about 3.1 mg/mL.

3. The method of claim 1, wherein the intravenous infusion is administered in less than 2 hours.

4. The method of claim 1, wherein the intravenous infusion is administered in about 1 hour.

5. The method of claim 1, wherein the intravenous infusion is administered in about 30 minutes.

6. The method of any one of claims 1-4, wherein the administrable solution is prepared by:
(a) dissolving lyophilized carmustine in sterile propylene glycol to form a reconstituted solution, wherein the amount of propylene glycol is 3 mL per 100 mg of carmustine; and
(b) diluting the reconstituted solution with an aqueous 0.9% sodium chloride solution or an aqueous 5% dextrose solution to obtain the administrable solution having a carmustine concentration of up to about 3.1 mg/mL.

7. The method of any one of claims 1-5, wherein the administrable solution has a carmustine concentration of no more than about 3.06 mg/mL.

8. The method of claim 1, wherein prior to dissolving the lyophilized carmustine in the propylene glycol, (i) the lyophilized carmustine and propylene glycol are stored in separate vials at 2-8° C. and (ii) the propylene glycol is allowed to attain room temperature just prior to dissolving the lyophilized carmustine in the propylene glycol.

9. The method of claim 1, wherein prior to dissolving the lyophilized carmustine in the propylene glycol, (i) the lyophilized carmustine and propylene glycol are stored in separate vials at 2-8° C. and (ii) the vials are allowed to attain room temperature just prior to dissolving the lyophilized carmustine in the propylene glycol.

10. The method of claim 7 or 8, wherein the propylene glycol is aseptically removed from its vial with a sterile syringe having a needle below 22 gauge and injected into the vial containing the lyophilized carmustine.

11. The method of any one of claims 1-9, wherein the reconstituted solution contains at least 90% of the initial carmustine after storage at 2-8° C. for up to 480 hours.

12. The method of any one of claims 6-10, wherein
the reconstituted solution is stored at 2-8° C. for up to 480 hours prior to step (b), and
after storage at 2-8° C. and prior to performing step (b), the reconstituted solution is examined for crystal formation and if crystals are observed, they are re-dissolved by warming the reconstituted solution to room temperature with agitation.

13. The method of any one of claims 6-11, wherein
the reconstituted solution is stored at 2-8° C. for up to 24 hours or at room temperature for up to 8 hours and protected from light prior to step (b),
optionally, after storage and prior to performing step (b), the reconstituted solution is examined for crystal formation and if crystals are observed, they are re-dissolved by warming the re-constituted solution to room temperature with agitation, and
the administrable solution is stored under normal room fluorescent light at 2-8° C. for up to 24 hours and subsequently at room temperature for up to 6 hours prior to administration by intravenous infusion.

14. The method of any one of claims 1-13, wherein step (b) is performed within 48 hours of the reconstituted solution being prepared.

15. The method of any one of claims 1-14, wherein administrable solution has a pH of about 4.2 to 4.8 and an osmolarity in the range of about 1900 to about 2000 mOsmol/L.

16. The method of any one of claims 1-15, wherein the patient is administered about 300 mg/m² to about 800 mg/m² carmustine.

17. The method of any one of claims 1-15, wherein the rate of administration of the intravenous infusion is no more than 26.6 mg/m²/min.

18. The method of any one of claims 1-15, wherein the rate of administration of the intravenous infusion is no more than 13.3 mg/m²/min.

19. The method of any one of claims 1-18, wherein the patient receives carmustine in a regimen with etoposide, cytarabine, and melphalan.

20. The method of claim 19, wherein the patient receives carmustine, etoposide, cytarabine, and melphalan as a conditioning regimen for autologous hematopoietic cell transplantation.

21. The method of any one of claims 1-19, wherein the patient suffers from relapsed or refractory non-Hodgkin lymphoma or Hodgkin lymphoma.

22. A method for administering carmustine to a patient in need thereof comprising administering by intravenous infusion over about 30 minutes to about 1 hour an administrable solution of carmustine, wherein
the administrable solution is prepared from a kit comprising a product vial containing 200 mg to 600 mg of lyophilized carmustine and a diluent vial containing 6-18 mL of sterile propylene glycol, the kit being stored at 2-8° C., and the administrable solution is prepared by:
(a) allowing the diluent vial to attain room temperature,
(b) aseptically removing the propylene glycol from the diluent vial, injecting it into the product vial containing lyophilized carmustine, and shaking the product vial to dissolve the lyophilized carmustine to form a reconstituted solution, wherein the amount of propylene glycol injected in the product vial is 3 mL per 100 mg of carmustine;
(c) optionally, storing the reconstituted solution and prior to performing step (d), the stored reconstituted solution is examined for crystal formation and if crystals are observed, they are re-dissolved by warming the reconstituted solution to room temperature with agitation, and
(d) diluting the reconstituted solution with an aqueous 0.9% sodium chloride solution or an aqueous 5% dextrose solution to obtain the administrable solution, wherein the concentration of carmustine in the administrable solution is up to about 3.1 mg/mL.

23. The method of claim 22, wherein the concentration of carmustine in the administrable solution is up to about 3.06 mg/mL.

24. The method of claim 22 or 23, wherein the intravenous infusion is administered in about 1 hour.

25. The method of claim 22 or 23, wherein the intravenous infusion is administered in about 30 minutes.

26. The method of any one of claims 22-25, wherein the patient receives carmustine in a regimen with etoposide, cytarabine, and melphalan.

27. The method of claim 26, wherein the patient receives carmustine, etoposide, cytarabine, and melphalan as a conditioning regimen for autologous hematopoietic cell transplantation.

28. The method of any one of claims 22-27, wherein the patient suffers from relapsed or refractory non-Hodgkin lymphoma or Hodgkin lymphoma.
